(19) United States
(12) Patent Application Publication
(10) Pub. No.: US 2010/0016546 A1
(43) Pub. Date: Jan. 21, 2010

(54) Therapeutic Peptide-Polysaccharide Biomaterials

(76) Inventor: Barney Bishop, Annandale, VA (US)

Correspondence Address: GEORGE MASON UNIVERSITY;OFFICE OF TECHNOLOGY TRANSFER, MSN 5G5, 4400 UNIVERSITY DRIVE, FAIRFAX, VA, 22030, US

(21) Appl. No.: 11/556,366

(22) Filed: Nov. 3, 2006

Related U.S. Application Data
(60) Provisional application No. 60/732,673, filed on Nov. 3, 2005.
(60) Provisional application No. 60/734,293, filed on Nov. 8, 2005.

(51) Int. Cl. C07K 9/00

(52) U.S. Cl. 530/322

(57) ABSTRACT

Peptide-polysaccharide conjugates may be created by linking at least one peptide to a polysaccharide. The peptide may be a defensin having at least a portion of the amino acid sequence of human-β-defensin-3. The portion may be the last 10-14 residues of the amino acid sequence. The polysaccharide may be functionalized with at least one R group prior to the linkage to aid in the linkage.

### CROSS REFERENCE TO RELATED APPLICATIONS

The present application claims the benefit of provisional patent application Ser. No. 60/732,673 to Bishop, filed on Nov. 3, 2005, entitled “Therapeutic Polysaccharide-Peptide Biomaterials,” and provisional patent application Ser. No. 60/734,293 to Bishop, filed on Nov. 8, 2005, also entitled “Therapeutic Polysaccharide-Peptide Biomaterials,” which are both hereby incorporated by reference.

### BRIEF DESCRIPTION OF THE DRAWINGS

FIG. 1 shows an example of a peptide-polysaccharide conjugate.

FIG. 2 shows a monosaccharide unit of cellulose.

FIG. 3 shows a monosaccharide unit of agarose.

FIG. 4 shows an example of reductive amination.

FIG. 5 shows an example of conjugating a functionalized polysaccharide with a peptide.

FIG. 6 shows another example of conjugating a functionalized polysaccharide with a peptide.

FIG. 7 shows an example of a flow diagram of creating a peptide-polysaccharide conjugate.

FIG. 8 shows another example of a flow diagram of creating a peptide-polysaccharide conjugate.

FIG. 9 shows yet another example of a flow diagram of creating a peptide-polysaccharide conjugate.

FIG. 10 shows the amino acid sequence of HBD-3.

FIG. 11 shows a chromatogram of analytical HPLC run of HBD-3₍₃₆₋₄₅₎ peptide before purification.

FIG. 12 shows a chromatogram of prep HPLC run of HBD-3₍₃₆₋₄₅₎ peptide.

FIG. 13 shows a chromatogram of analytical HPLC run of HBD-3₍₃₆₋₄₅₎ peptide after purification.

FIG. 14 shows a MALDI-TOF result of HBD-3₃₆₋₄₅.

FIG. 15 shows a MALDI-TOF result of HBD-3₃₆₋₄₅(Rev2).

FIG. 16 shows an example of periodate oxidation of cellulose.

FIG. 17 shows results antimicrobial activity of free and conjugated HBD-3₃₂₋₄₅ peptide.

FIG. 18 shows results of colony counts for peptide HBD-3₃₂₋₄₅ and peptide HBD-3₃₆₋₄₅ in tabular form.

FIG. 19 shows results of colony counts for peptide HBD-3₃₆₋₄₅(Rev 2) and peptide-polysaccharide conjugate HBD-3₃₆₋₄₅(Rev2)-agarose conjugate in tabular form.

### DETAILED DESCRIPTION OF THE INVENTION

The claimed invention relates to engineering antimicrobial biomaterials that can provide the basis for therapeutics for the treatment of infection relating to the digestive track, such as gastric infection, enteric infection and gastrointestinal infection. In particular, the antimicrobial biomaterials involve utilizing peptide-polysaccharide and polysaccharide-peptidomimetic materials to create antimicrobial constructs (i.e., peptide-polysaccharide conjugates) that can be adapted for such therapeutic use.

### I. Introduction

A. Gastrointestinal Pathogens

Gastrointestinal (GI) infections comprise a variety of invasive diseases and infections that can affect the GI tract and other tissues. Common infections that can be caused by illness, diet changes and pathogens. Examples of GI pathogens include viruses, bacteria, parasites and fungi. While many of these illnesses are treatable, they can have fatal consequences if contracted and left untreated, especially for children. For instance, rotavirus is a significant cause of diarrhea all over the world, especially in small children. Transmission is likely through fecal-oral and aerosol. Generally, rotavirus accounts for the deaths of many children in the first 2 to 4 years of life. Another common cause of GI infection in children is cryptosporidium parvum. It can be spread from person to person and from animal to person by direct contact or by contaminated water. Some strains of E. coli with specific properties are also associated with diarrhea disease too.

The widespread increase of pathogens developing resistance to currently used antibiotics presents a growing problem in treating GI infections and other infections. Many GI pathogens have demonstrated the ability to mutate and gain resistance to many of the antibiotics currently in use. Therefore, there is an urgent need to develop new therapeutics.

According to the National Institute of Allergy and Infectious Disease, the Center for Disease Control has estimated that 76 million people suffer from foodborne illnesses, in which as many as 325,000 people are hospitalized and over 5,000 die per year. In 1994, there was an outbreak of Salmonella enteriditis in the United States that affected approximately 224,000 people. This outbreak was traced to a tanker shipment of contaminated liquid ice cream. An outbreak of Salmonella typhimurium (“S. typhimurium”) in Illinois in 1985 affected over 170,000 people as a result of contaminated milk. What made this outbreak particularly disturbing was the fact that the strain of S. typhimurium involved demonstrated resistance to nine different antibiotics.

The issue of GI pathogens and the safety of food supplies tend to be further complicated by the threat of bioterrorism. The majority of work involving countering and responding to biological weapons has primarily focused on the airborne delivery of pathogens and associated respiratory infections. Such an attack can result in maximum impact on the battlefield. However, bioterrorists may opt for an alternative target, namely the food supply. Contaminating the food supply with a biological agent may lead to widespread panic and civil disorder.

Addressing the use of biological agents to intentionally contaminate the food supply has not yet been a priority, albeit the threat is real. For instance, this type of attack occurred in the United States in 1984, when a religious cult introduced S. typhimurium into a salad bar in Oregon. This introduction caused over 700 people to contract salmonellosis.

Some examples of pathogens of greatest concern are listed below in TABLE 1.

TABLE 1
Examples of Gastrointestinal Pathogens

| Pathogen | Associated Illness |
|---|---|
| Salmonella typhi | Acute fever; diarrhea; potential intestinal |
| | rupture |
| Shigella dysenteriae (type 1) | Dysentery (up to ~20% fatality) |
| Escherichia coli (0157:H7) | Acute hemorragic diarrhea; possible long |
| | term problems |
| Bacillus anthracis | Ulceration; fever and abdominal pain; |
| (referring to gastrointestinal | blood-tingled stool; vomit; potentially |
| anthrax infections) | fatal |
| Vibrio cholerae | Severe diarrhea (up to ~50% fatality) |

B. Antimicrobial Peptides

Explosive increases in the number of bacterial pathogens demonstrating resistance to antibiotics and the threat of bioterrorism have led researchers to explore alternative approaches to combating infection. Using antimicrobial peptides to combat GI pathogens may offer a solution to these problems. Antimicrobial peptides are part of the innate immune system. Higher organisms have evolved defense mechanisms based on a diverse array of antimicrobial peptides to combat infection and pathogens. These peptides are essential components of the innate immune system in mammals and other higher organisms, including humans. Many of these peptides demonstrate a broad spectrum of antimicrobial activity against bacteria, fungi and viruses. It has been suggested that the role of antimicrobial peptides in combating microbes is as important as other elements of immunity, such as antibodies, macrophages, neutrophils and lymphocytes. Production and secretion of cationic antimicrobial peptides are often induced by antigens associated with invading microbes. Antimicrobial peptides represent an ancient defense mechanism, one that has likely been around for billions of years. Yet, pathogens have not developed widespread resistance to them. Therefore, antimicrobial peptides are an area of great interest in the quest to develop new antibiotics.

While debate exists regarding the details of antimicrobial peptides, it is generally believed that antimicrobial peptides derive at least part of the antimicrobial properties from direct interactions with membranes of bacteria. Most theorists agree that the antimicrobial peptides, which tend to be rich in basic residues, are electrostatically drawn to microbial membranes. The outer surfaces of bacterial membranes are often rich in lipids with negatively-charged head groups, resulting in the outer surface having an overall anionic character. The outer surfaces of eukaryotic membranes tend to be rich in lipids with neutral head-groups. Therefore, bacterial membranes are preferentially targeted by the cationic antimicrobial peptides. The initial electrostatic interactions between the peptide and the bacterial membrane are believed to lead to the disruption of membrane integrity. In some cases, it is believed that the peptides are inserted into the membrane and form pores. In others, the peptides may permeablize the membrane and disrupt the integrity without forming discrete pores.

Antimicrobial peptides are generally small (approximately 12-50 amino acids in length) with overall cationic and amphiphilic character. They are known to demonstrate antimicrobial activity against both bacteria and fungi. One major type of antimicrobial peptides include defensins.

C. Defensins

Defensins are a family of potent antimicrobial peptides produced by various types of cells in the body, including but not limited to neutrophils, macrophages, epithelial cells and leukocytes. These small antimicrobial peptides are a major component of the first line of defense against invading pathogens. They are known to demonstrate broad antimicrobial activity against bacteria, fungi, parasites and viruses. Like other cationic antimicrobial peptides, defensins are believed to function by binding to the membranes and increasing membrane permeability of those microbes.

In mammals, there are three main families of defensins, namely α-defensins, β-defensins and θ-defensins, that share some common features. They are generally rich in basic amino acid residues, such as arginine and lysine. In α-defensins and β-defensins, six cysteine residues form three intramolecular disulfide bonds, stabilizing the 3D structure that contains a triple-stranded antiparallel β-sheet. α-defensins are produced primarily in neutrophils and small intestinal paneth cells. β-defensins are produced in leukocytes and epithelial cells. Four distinct β-defensins have been identified in human, namely human β-defensin-1 (“HBD-1”), human β-defensin-2 (“HBD-2”), human β-defensin-3 (“HBD-3”) and human β-defensin-4 (“HBD-4”). θ-defensins are short peptides, which are produced by rhesus macaques. Unlike other defensins, θ-defensins are unique in that they are cyclic peptides, where the N- and C-termini have been linked through a backbone peptide bond.

D. Antimicrobial Biomaterials and Polysaccharides

Generally, peptides and proteins are not well suited for oral delivery because they are quickly broken down by enzymes in the digestive tract. Peptidomimetics, which are resistant to enzymatic proteolysis, may provide a possible solution to this problem. However, the utility of antimicrobial peptidomimetics can be hampered by lower potency and increased hemolytic activity relative to their peptide counterparts. There is also the possibility that they may be absorbed and enter the blood stream. Conjugating and/or functionalizing antimicrobial peptides and mimetics to polysaccharides may provide a solution to these problems.

It should be noted that conjugation and functionalization have different meanings. Conjugation refers to the linking of a peptide to a polysaccharide. Functionalization refers to the introduction of functional groups (such as aldehyde groups) to a polysaccharide.

A similar strategy has been used to improve the effective serum lifetimes of therapeutic proteins by conjugating them to a biocompatible polymer, such as polyethylene glycol. A polysaccharide scaffold (such as cellulose, etc.) may function similarly by shielding attached peptides and proteins from proteolysis. Additionally, displaying antimicrobial groups on a polysaccharide scaffold should increase their effective local concentration and thereby increase antimicrobial potency, thereby allowing the use of slightly less potent mimetics that do not demonstrate hemolytic activity.

Polysaccharide conjugates having polysaccharides that tend not to be absorbed into the bloodstream may be suitable for the treatment of GI infections. Cellulose is a dietary fiber, which is not broken down by human digestive enzymes and is not absorbed. Therefore, it is reasonable to assume that the cellulose core of cellulose-peptide/mimetic conjugates will also be resistant to human digestive processes and will remain localized in the GI tract, the site of infection. Moreover, the close proximity of peptides attached to the scaffold can allow several peptide groups to simultaneously attach a single bacterium, thus increasing effective local concentration of the peptide. These engineered biomaterials can be ideally suited for the treatment of GI infection.

### II. Embodiments

A. Peptide-Polysaccharide Constructs

Generally, antimicrobial peptides, peptide fragments and mimetics can be linked to polysaccharides (e.g., derivatized dietary fibers) that are not readily absorbed during digestion. The modular nature of the proposed biomaterials tends to make them versatile antimicrobial agents. Alternate polysaccharide scaffolds can be used to alter the biophysical and solubility properties on the conjugates. The degree of loading and the nature of the peptides can be altered in order to fine tune the specificity and potency of the conjugates. Careful selection of the polysaccharide scaffold and control of substitution should make it possible to produce materials that are restricted to the gastrointestinal space and that are not absorbed into the blood stream. Such strategy can reduce the risk of possible toxic effects while localizing the drug to a particular area of interest. Some of the chemistry used to assemble the peptide-polysaccharide/peptidomimetic conjugates and their components may be based on well-established chemical reactions and methods. However, the peptide-polysaccharide/peptidomimetic conjugates present a novel class of therapeutics. While some antimicrobial biomaterials have been reported, they were not intended to function as therapeutic agents.

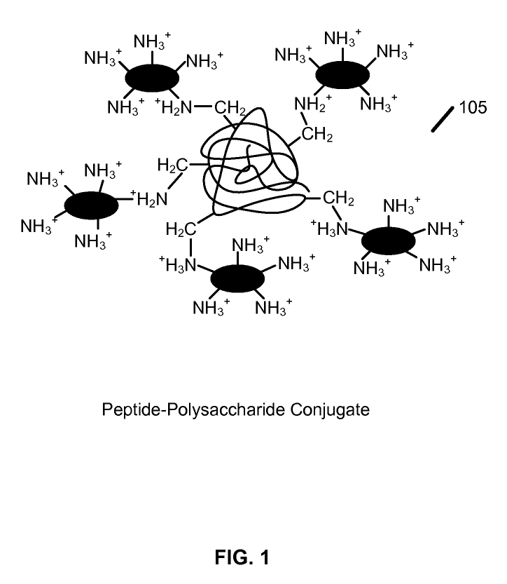

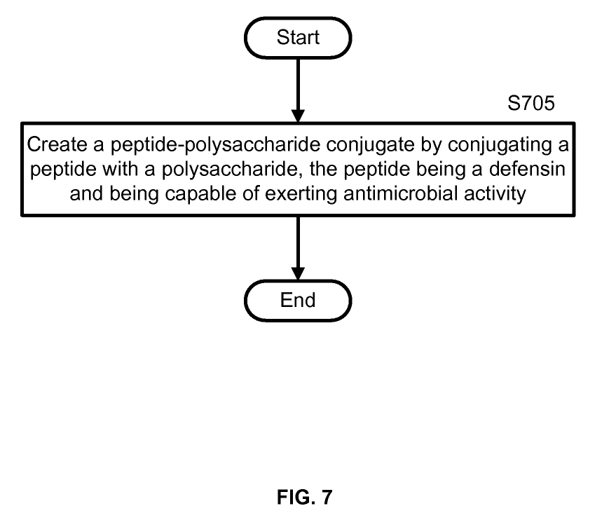

FIG. 1 shows an example of a peptide-polysaccharide conjugate 105. As an embodiment, as shown in FIG. 7, one or more peptides can be conjugated to a polysaccharide S705. The peptide(s) may be based on at least a portion of the amino acid sequence of a defensin, such as HBD-3. Where the defensin is HBD-3, the amino acid sequence portion may be the last 10-14 residues of the amino acid sequence. For instance, one portion may be KSSTRGRKSSRRKK. Another example may be RGRKSSRRKK. A yet another example may be RGRRSSRRKK with an amide group located on the C-terminus.

The terms “peptide-polysaccharide” and “polysaccharide-peptide” may be used interchangeably, where both refer to the combination of peptides and polysaccharide.

A polysaccharide may provide an ideal scaffold for an antimicrobial construct. Such construct may serve as therapeutics to combat GI pathogens. A plurality of polysaccharides may be used to formulate the peptide-polysaccharide conjugates.

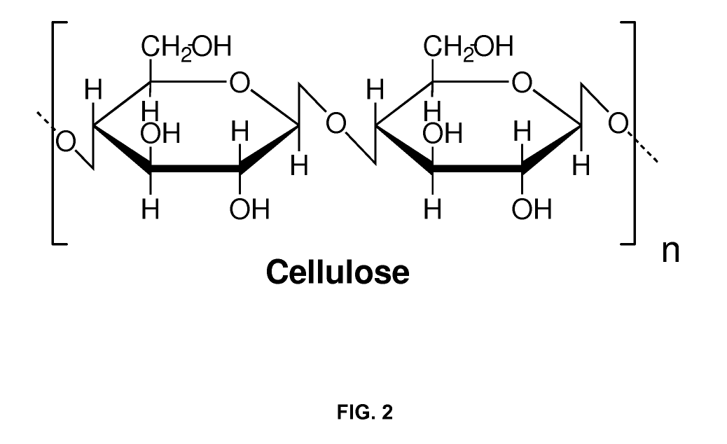

One example of a polysaccharide that can be used is cellulose. Cellulose is a polysaccharide that is not readily broken down and absorbed by human digestive processes. Similarly, a therapeutic polysaccharide conjugate will also likely remain localized in the GI tract and not readily enter the blood stream. As shown in FIG. 2, cellulose is a linear homopolymer of D-glucose units, where all of the sugar units are connected by β(1→4)-glycosidic bonds. Cellulose is the most abundant polysaccharide in the world, and is a major structural component in plant cell walls. Cellulose may have up to about 15,000 monosaccharide units, denoted as “n” in the figure. The abundant primary and secondary hydroxyl groups on polysaccharides can provide multiple points of attachment. One skilled in the art would understand that the functionalization and chemical modifications of cellulose are well known.

Another polysaccharide example is agarose (e.g., cross-linked agarose). Cross-linked agarose, as opposed to regular (non-cross-linked) agarose, may be easier to use simply because of its commercial availability. For instance, derivatized cross-linked agarose can be obtained from Pierce Biotechnology, Inc. (“Pierce”) of Rockford, Ill. It should be noted that this example is not intended to limit the practice of the claimed invention.

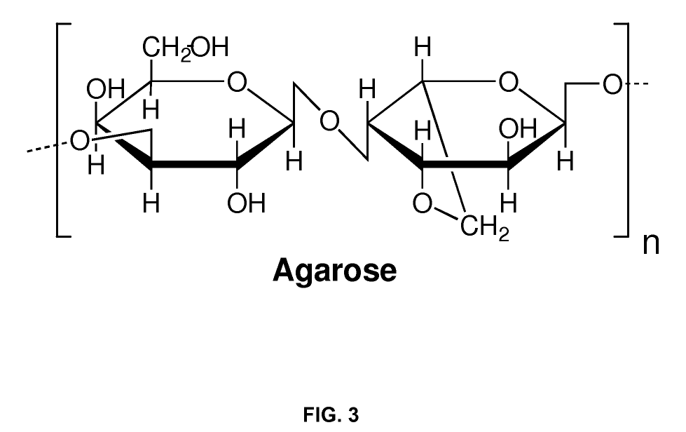

As shown in FIG. 3, agarose consists of D-galactose-β(1→4)3,6-anhydro-L-galactose repeat units linked by α(1→3) glycosidic bonds. It is an unbranched polysaccharide and is a major component of agar. Agarose may have up to about 1,000 monosaccharide unites, denoted as “n” in the figure.

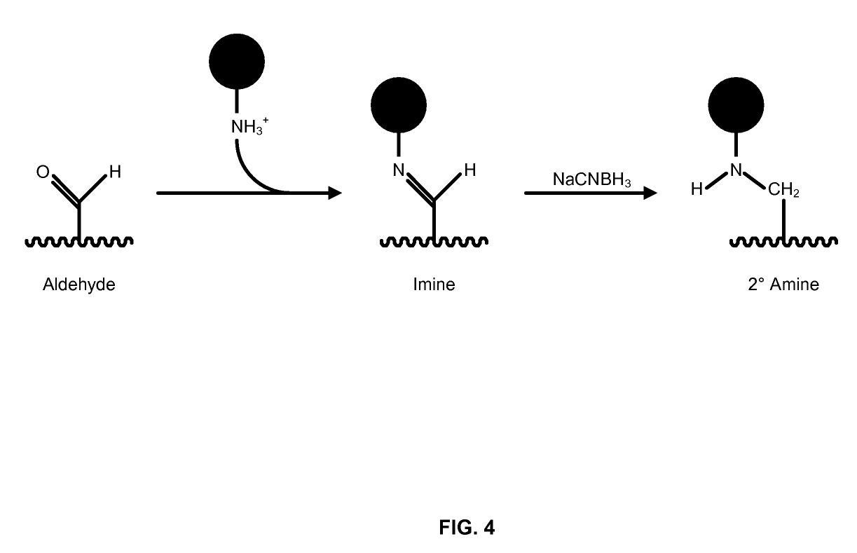

Antimicrobial peptide moieties 510 may be linked to the functionalized polysaccharide 505, as exemplified in FIGS. 4, 5 and 6. In one embodiment, where the polysaccharide is functionalized with aldehyde groups, the peptides may be linked to the functionalized polysaccharide 505 by formation of a Schiff base between aldehyde groups present on the functionalized polysaccharide 505 and the free amino groups of lysine side chains and the N-terminus of the peptide. The resulting imines may then be reduced to form more stable secondary amines using a reducing agent, such as sodium cyanoborohydride (NaCNBH₃).

Agarose that is cross-linked internally may be used as a surrogate for cellulose. However, it should be noted that cellulose is not likely to be cross-linked internally. Cross-linked agarose may utilize protein/peptide conjugation chemistry that is similar to that which can be used with cellulose constructs. It is likely to provide a model platform for experiments aimed at refining protocols for peptide conjugation, evaluating loading, antimicrobial potency, and hemolytic activity.

Cellulose and agarose differ at their glycosidic bonds. Cellulose is beta (1→4), and agarose is alpha (1→3). Furthermore, the agarose used herein was derivatized in order to introduce aldehyde groups onto it. These aldehyde groups are generally absent in cellulose and therefore must be introduced into cellulose. The presence of aldehyde groups is often important because they may be required for linking the peptide to the polysaccharide via reductive amination. Peptides tend to be linked to the polysaccharide scaffold via primary amino groups present on the peptide and reductive amination of the aldehyde groups.

In addition to cellulose and agarose, other polysaccharides may be used to practice the claimed invention. Nonlimiting examples include dextran, β-glucan, β-D-glucan, guar gum, arabinogalactin, alginate, pectin and methyl cellulose.

The peptide may have many sites where the polysaccharide can attach. Sites include, but are not limited to, the ε-amino group of lysine of said peptide, and the α-amino group of the N-terminus of said peptide.

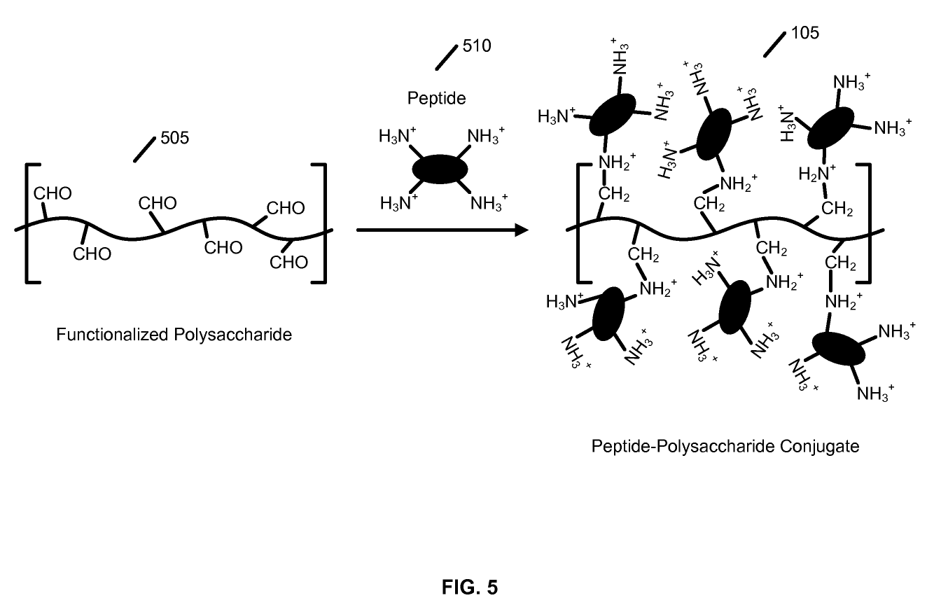

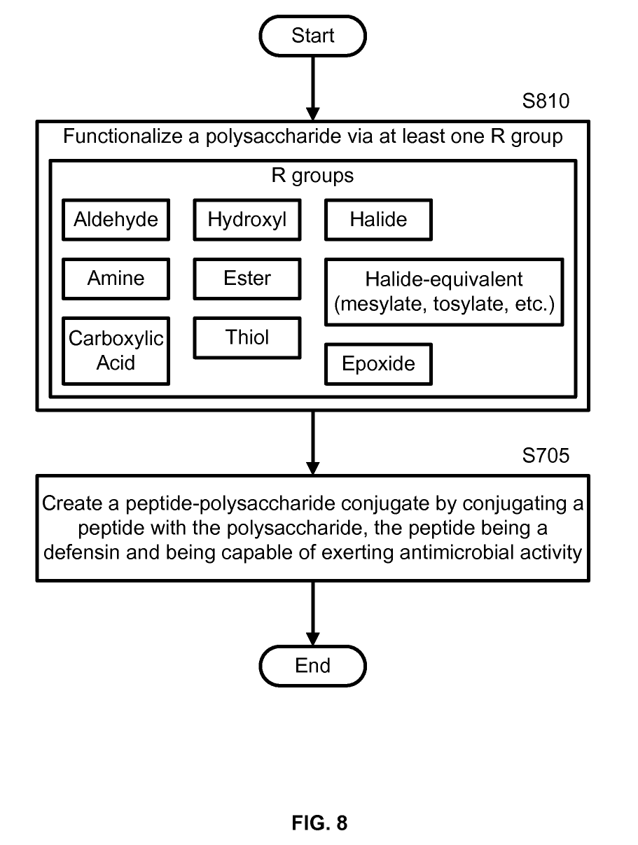

To help facilitate peptide attachment, the polysaccharide may need to be functionalized S810 prior to being linked with a peptide(s), as illustrated in FIGS. 5-6 and 8. A multitude of R groups may be selected to achieve functionalization S810. For instance, the R group may be any one or a combination of the following groups: aldehyde, amine, carboxylic acid, hydroxyl, ester, thiol, halide, and epoxide. Halide equivalents, such mesylate and tosylate, as well as both their derivatives, may also be used. It should also be noted that functionalization may be used interchangeably with derivatization (such as to refer to attaching aldehyde groups to polysaccharides).

As yet another embodiment, the peptide may be optionally synthesized. Synthesis may be achieved using a variety of procedures. One procedural example is using standard solid-phase peptide synthesis (“SPPS”) based on 9-fluoroenylmethoxycarbonyl (“Fmoc”) chemistry. Fmoc chemistry is an amine protection strategy that may be incorporated to prevent unwanted reactions at the α-amino group of the residue. In other words, the α-amino groups on amino acids may be provided temporary protection as they are being coupled. Generally, at least one cycle of SPPS should be conducted. Another example of peptide synthesis includes SPPS based on t-butoxycarbonyl (“Boc”) chemistry, which is another amine protection strategy.

Additionally, it may be desirable to purify the peptide prior to conjugation. A multitude of chromatographic methods may be used for purification. Nonlimiting purification examples include high performance liquid chromatography (“HPLC”), fast protein liquid chromatography (“FPLC”), etc. Columns for various chromatographic techniques for purifying proteins and/or peptides include reverse-phase, ion exchange, gel filtration/size exclusion/gel permeation, affinity, hydrophobic analogous column, etc.

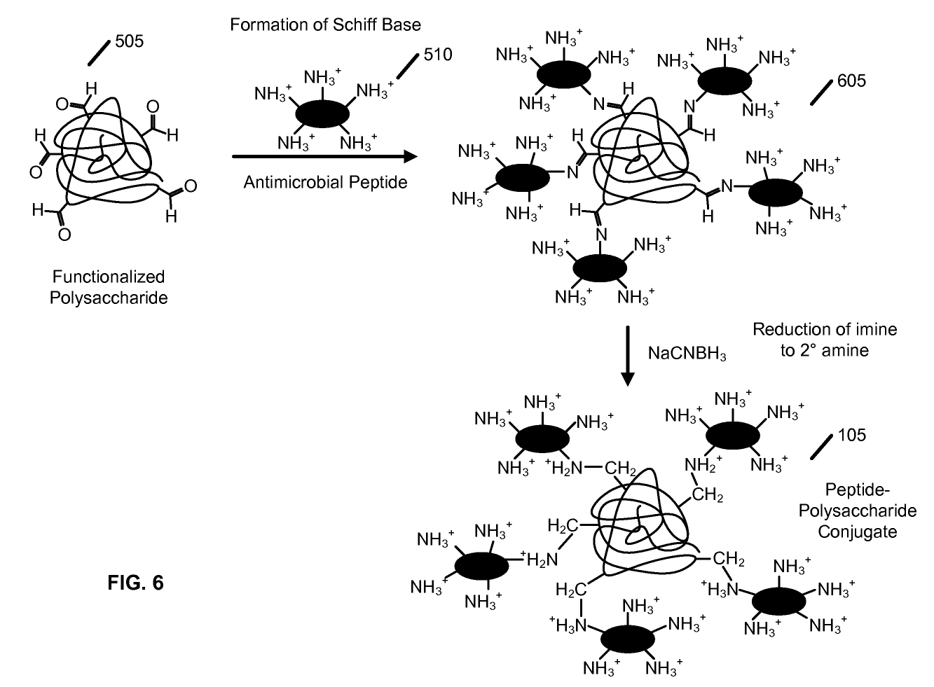

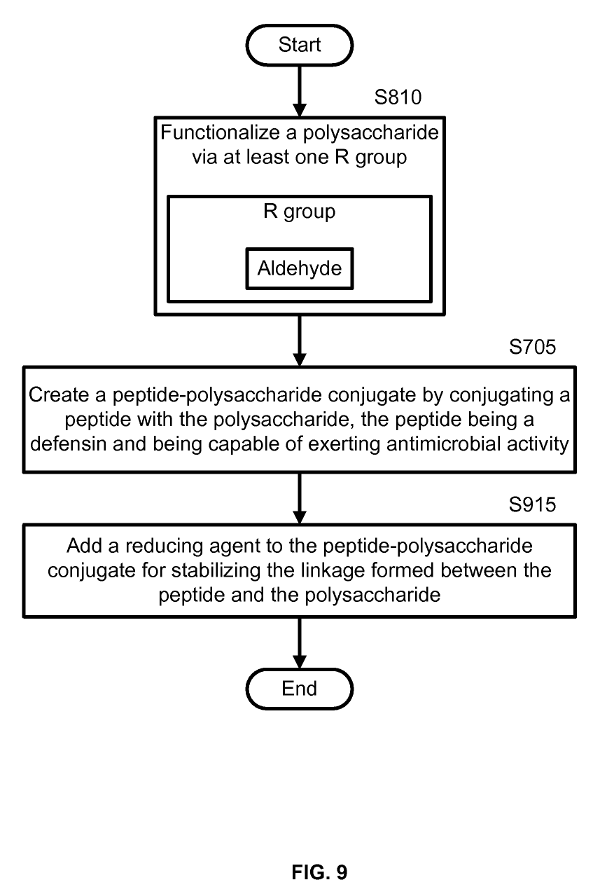

In one embodiment, the polysaccharide may be functionalized with aldehydes. Referring to FIGS. 6 and 9, Schiff base formation may occur between one or more aldehyde groups of the functionalized polysaccharide 505 and the peptide 510. The formation of Schiff bases may result in the peptide-polysaccharide intermediary 605 being connected via imine groups. However, this intermediary 605 may not be stable. Hence, to stabilize the linkage between the polysaccharide and peptide, reductive amination may be performed S915. Using a reducing agent (e.g., sodium cyanoborohydride, sodium triacetoxyborohydride, etc.), the imine groups may be converted into secondary amine groups. This conversion may result in the desired peptide-polysaccharide conjugate 105.

Reductive amination may be necessary where the R group on the functionalized polysaccharide is an aldehyde group. However, reductive amination may not be necessary where other functional groups are used because some functional groups tend not to be converted into imine intermediaries. Rather the peptide-polysaccharide conjugate formed by linking peptides with polysaccharides that are functionalized with other functional groups may directly form a stable link. For example, hydroxyl functional groups may be used to form ethers or esters. Also, amine functional groups may be used to form amide bonds. Additionally, carboxylic acid functional groups may be used to form esters or amides. Furthermore, epoxides, esters, halides and halide equivalents may be displaced through nucleophilic attacks. Moreover, thiols may be used to form disulfide bonds or thioethers.

B. Antimicrobial Peptides and Mimetics

The following embodiments serve as models to represent the various antimicrobial peptides and mimetics that are known for their potency and broad antimicrobial activity. As hoards of antimicrobial peptides and mimetics exist, this list is intended to exemplify those that can be used to create constructs, and in no way does this listing limit which antimicrobial peptides and mimetics can be used to create constructs.

For purposes of exemplifying how constructs can be made, attention is now turned towards the use of HBD-3, HBD-3 fragments, and antimicrobial β-peptides (a peptide mimetic).

1. HBD-3

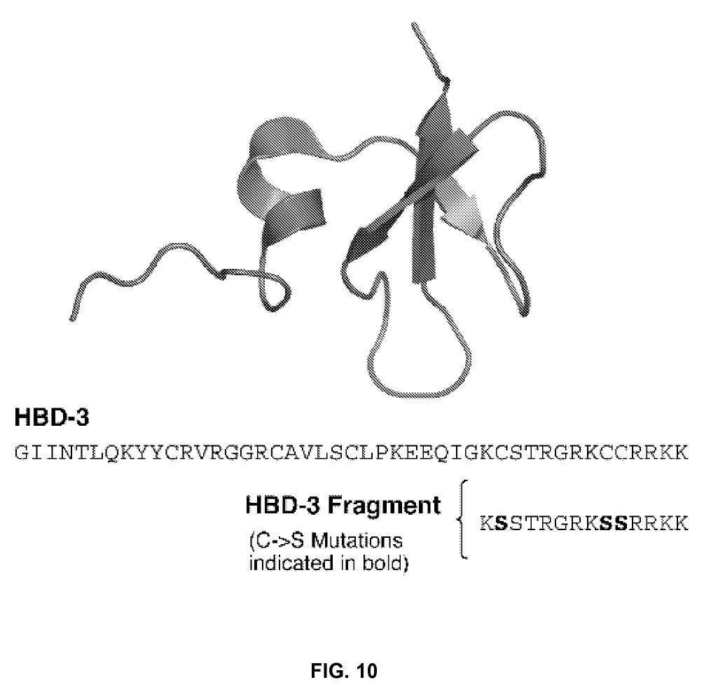

β-defensins are potent antimicrobial peptides and are also essential elements of innate immunity in mammals. These peptides share a common three-dimensional configuration. Additionally, they demonstrate a broad range of potencies and specificities, such as killing bacteria, fungi, and viruses. Having 45 amino acids in length, as illustrated in FIG. 10, HBD-3 is arguably the most potent of the human β-defensins, killing both Gram positive and Gram negative bacteria. Peptides based on the amino acid sequence of the C-terminal region of HBD-3, namely a 14-residue peptide based on residues 32-45 of HBD-3 (“HBD-3₃₂₋₄₅”) and a 10-residue peptide based on residues 36-45 of HBD-3 (“HBD-3₃₆₋₄₅”), show high antimicrobial activity. Yet, HBD-3₃₂₋₄₅ is often slightly more potent than HBD-3₃₆₋₄₅. Even when denatured, HBD-3 (in contrast with other β-defensins) still tends to be antimicrobial potent.

2. HBD-3 Fragments

Even fragments of HBD-3, have demonstrated antimicrobial activity. For example, a peptide corresponding to the 14 C-terminal residues of HBD-3, also illustrated in FIG. 10, had potent antimicrobial activity against E. coli (1±0.1 mg/ml). While this peptide tends not to have as broad as a spectrum of activity as the parent HBD-3, this peptide's size makes it synthetically accessible. In essence, the size alone may make the child peptide a good basis for future peptide libraries.

3. Antimicrobial β-Peptides

The design of antimicrobial β-peptides, which are peptides composed entirely of β-amino acids, are well known in the art. These peptides were designed to reproduce the spatial distribution of polar and hydrophobic side-chains present in naturally occurring antimicrobial peptides, such as magainins and cecropins. With some refinement of the sequences, they were able to generate a family of β-peptides with decent potency against E. coli (MIC 9-26 μg/ml). These peptide mimics are well suited for incorporation into the proposed constructs. The reported antimicrobial β-peptides are synthetically accessible, and a range of appropriately protected β-amino acids are commercially available. These β-peptides can also provide a basis for future peptide mimetic libraries.

C. Peptide Synthesis

As an embodiment, peptides and peptide mimetics may be assembled using SPPS based on Fmoc chemistry. As another embodiment, SPPS based on Boc chemistry may be used to synthesize peptides.

In peptide synthesis, the peptide may be assembled one amino acid at a time. For instance, using SPPS based on Fmoc chemistry, the first amino acid is attached to an insoluble resin through its carboxyl terminus. This chemistry tends to utilize an orthogonal protection scheme, where amino acid α-amino groups can be protected with the base-labile Fmoc group and where sensitive side-chain groups can be protected with base-stable-acid-labile protecting groups.

SPPS is a stepwise process involving deprotection, washing and coupling cycles followed by cleavage from the resin. SPPS involves binding a peptide that is to be synthesized to a solid support, such as cross-linked polystyrene. Amino acids may be added in a stepwise fashion, which is usually one amino acid per cycle, until the complete sequence has been assembled. Each cycle includes a deprotection step, washes following deprotection, a coupling step and more washes following coupling. The cycle is often repeated for each amino acid residue. Since the peptide being synthesized remains bound to the resin, reagents and soluble byproducts can be removed after each coupling and deprotection step. Removal of acid-labile protecting groups may be achieved under conditions used to cleave the peptide from the resin.

The larger HBD-3, at 45 amino acids, is generally too large to be synthesized manually. However, wild-type HBD-3 is commercially available (e.g., acquirable from Peptides International, Inc.) and may be synthesized.

In one embodiment, peptides corresponding to the C-terminal domain of HBD-3 may be synthesized. As shown in TABLE 2, HBD-3₃₂₋₄₅ and HBD-3₃₆₋₄₅ may be assembled on Wang resin, which provides peptides with a free C-terminal carboxyl group after cleavage. For example, HBD-3₃₂₋₄₅ peptide may be synthesized at 0.525 mmol scale using 0.833 g of resin with a loading of 0.63 mmol/g. In another example, HBD-3₃₆₋₄₅ may be synthesized at 0.47 mmol scale using 1.000 g of resin with a loading of 0.47 mmol/g (4-alkoxybenzyl alcohol linker) using Fmoc chemistry. Fmoc should bind to the N-terminal of the peptide to protect that side of the peptide from unwanted reactions.

TABLE 2
Examples of Peptides on C-terminal region of HBD-3

| Name | Polysaccharide | Net Charge at pH 7 |
|---|---|---|
| HBD-3₃₂₋₄₅ | KSSTRGRKSSRRKK | +8 |
| HBD-3₃₆₋₄₅ | RGRKSSRRKK | +7 |
| HBD-3₃₆₋₄₅(Rev2) | RGRRSSRRKK-NH₂ | +8 |

Once synthesized, the N-terminal Fmoc group may be removed via multiple washing cycles. For instance, deprotection may be achieved by three cycles of 2 min washes of the resin with 10 ml of 20% piperidine and 80% N,N-dimethyl formamide (“DMF”). Three one-minute washes (using e.g., DMF, 10 ml) between the deprotection and coupling steps may be necessary to remove byproducts and unreacted reagents.

As applied to the exemplified HBD-3₃₆₋₄₅, the resin may be suspended in 10 ml of dichloromethane (“DCM”) and agitating the resin for some time (e.g., 1 min). Agitation may cause the resin to swell. After suspension and agitation, the resin can be drained. This process may be repeated (e.g., 3 times). In a similar fashion, the resin may also be cyclically washed (e.g., 3 one-minute washes) using 10 ml of N,N-Dimethylformamide (“DMF”). Washing with DMF between the deprotection and coupling steps may be necessary to remove byproducts and unreacted reagents. The base-labile α-amino protecting Fmoc group may be removed using a 20% mixture of piperidine in DMF.

Coupling may involve the addition of a protected amino acid through formation of a peptide bond between the carboxyl group of the amino acid and the free N-terminus of the growing peptide. Coupling cocktails can be made by combining the amino acid, 2-chloro-N-hydroxy-benzotriazole (“Cl—HOBt”), 1H-benzotriazolium 1-[bis(dimethylamino)-methylene]-5-chloro-(1-),3-oxide hexafluorophosphate (“HCTU”) and N,N-Diisopropyl-ethylamine (“DIEA”). Referring to the exemplified HBD-3₃₆₋₄₅, coupling steps involve combining (a) the Fmoc-amino acid (e.g., 1.41 mmol) and the activating reagents Cl—HOBT (˜1.41 mmol, ˜0.191 g), HCTU (˜1.36 mmol, ˜0.564 g) and DIEA (˜3.53 mmol, ˜0.603 ml), and (b) dissolving in N-methylpyrrolidone (10 ml). To improve coupling efficiency, an excess of protected amino acid and coupling reagents may be used.

Coupling times can range from ˜30 min to ˜1 hr. Coupling and deprotection reaction efficiency may be monitored by the ninhydrin test (“Kaiser test”), which detects the presence of free primary amines. In cases where incomplete or inefficient coupling occurs, conditional double-coupling or repetitive coupling, and acetylation may be used to maximize final yield, coupling efficiency and ease of purification.

Once the sequence of a peptide has been assembled, the resin may be dried and weighed. In the case of HBD-3₃₆₋₄₅, the dried resin after synthesis weighed 1.66 g, indicating a weight gain of 0.66 g. The peptide sequence may be cleaved from the resin using a mixture comprising anhydrous trifluoroacetic acid (“TFA”), triisopropylsilane (“TIS”) and water. In one embodiment, the cleavage mixture comprises 95:2.5:2.5 (v/v) TFA/TIS/water. TIS and water may serve as scavengers for reaction byproducts. The combined volume of this mixture (for instance, 10 ml) may be combined with the resin and allowed to stir for some time (e.g., 3 hr) at room temperature. After the desired time has elapsed, the peptide and resin may be recovered by precipitation using, for example, a 10-fold (e.g., 100 ml) excess of cold ether followed by filtration. The precipitation reaction may be allowed to sit, for instance, at 0° C. for 1 hr. The resin-peptide mixture may be washed twice with 5 ml aliquots of cold ether and then dried under vacuum. The peptide may then be dissolved in water or 0.1% TFA in water and separated from the resin by filtration.

Antimicrobial activity of these HBD-3 peptides may be evaluated using E. coli BL21(DE3). Examples of protocols that may be used are described herein.

D. Peptide Purification

Following cleavage from the resin, these peptides may be isolated and/or purified. One purification method that may be used includes reverse-phase HPLC. Another purification method includes FPLC.

Synthesized peptides may be purified by reversed-phase HPLC with an Altech Altima C18 (preparative column: 250 mm×4.6 mm, analytical column: 250 mm×10 mm). Solvents used for the Altech Altima C18 include solvents A and B. Solvent A may comprise of 0.1% TFA in water. Solvent B may comprise of 80% acetonitrile (“ACN”) with 0.08% TFA. Solvents may be filtered to remove particulate matter and for partial degassing.

In one embodiment, purification of HBD-3₃₂₋₄₅ may begin with the small-scale analysis of crude peptide using a gradient of 12-27% solvent B over 30 min at a flow rate of 0.7 ml/min. A preparative scale run may be performed using the 12-27% solvent B gradient over 30 min, at a flow rate of 3.5 ml/min with fractions being collected at 1-minute intervals. The elution of peptide may be monitored by absorbance at 214, 220 and 230 nm. The peptide may be determined to have eluted at approximately 12 min. To verify the purity of fraction eight, an analytical run of the fraction using a 10-20% solvent B gradient was performed. Mass spectroscopy may also be used to verify the presence of the peptide in the fraction. This process may afford approximately 39 mg of purified peptide.

In another embodiment, HBD-3₃₆₋₄₅ may be purified using the analytical and preparative columns and buffers (as previously described) with a modified gradient. Small-scale analysis of crude HBD-3₃₆₋₄₅ using a gradient of 8-20% solvent B over 30 min at a flow rate of 0.7 ml/min may be carried out. The peptide may be isolated using a similar gradient at a flow rate of 3.5 ml/min with fractions collected at 1-minute intervals. The elution of peptide may be monitored by absorbance at 214, 220 and 230 nm. The peptide may be determined to have eluted over approximately 11-13 min. To verify the purity of the isolated fraction, analytical HPLC using 8-20% solvent B gradient may be performed. A single peak may be observed.

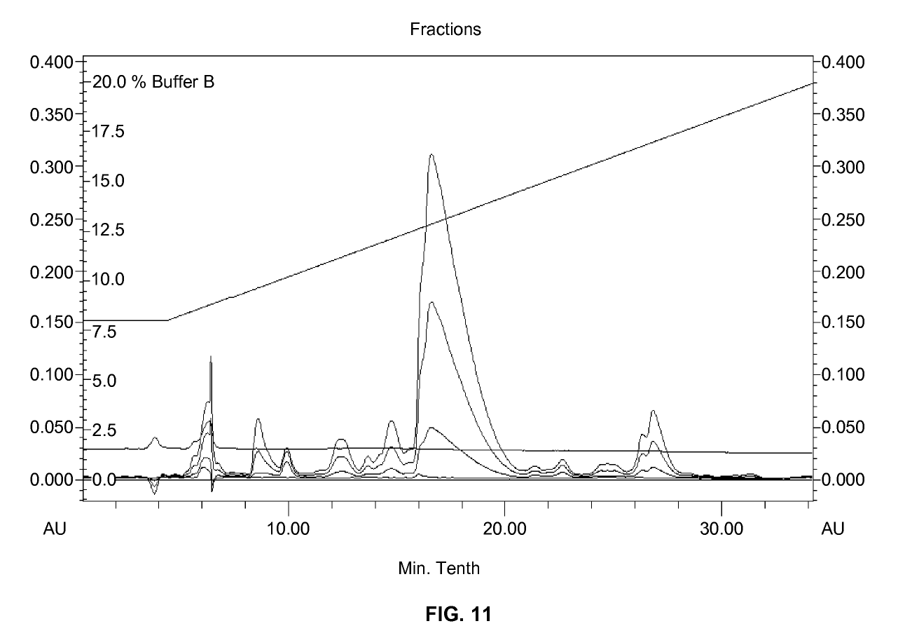

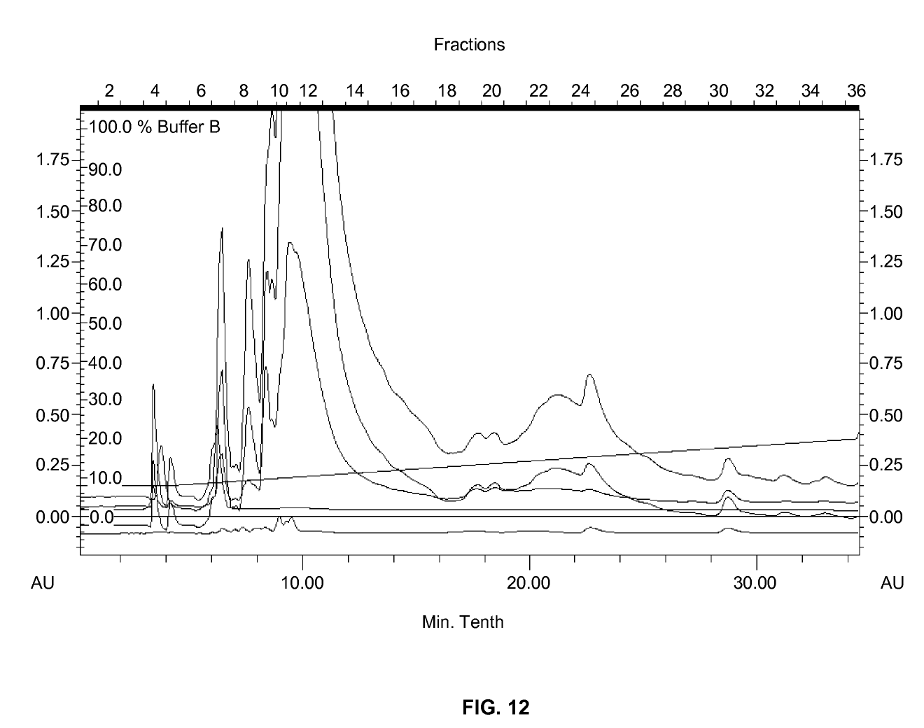

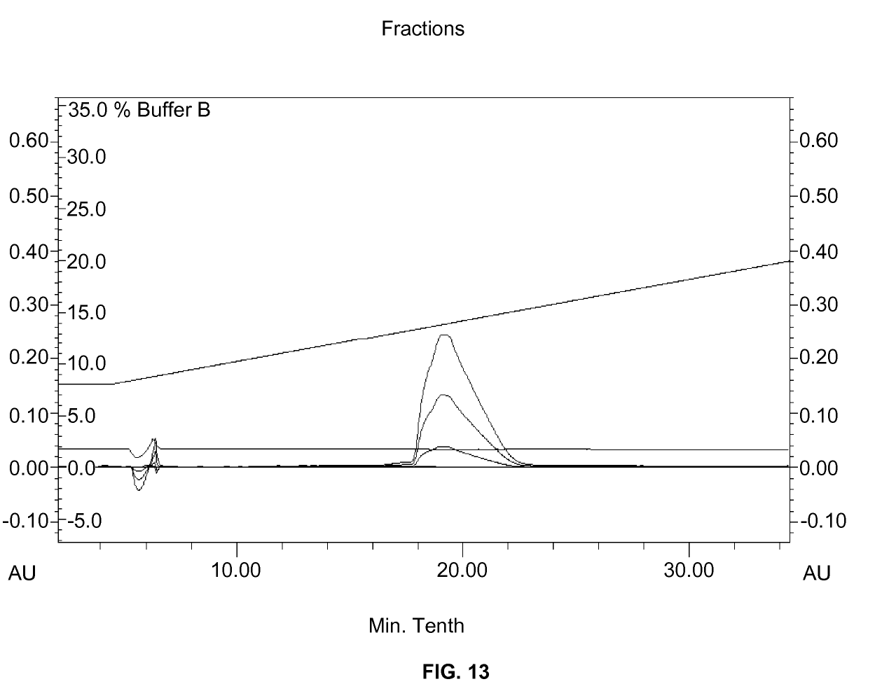

The following chromatograms illustrate comparisons of HBD-3₃₆₋₄₅ before and after purification. FIG. 11 shows a chromatogram of an analytical HPLC run of HBD-3₃₆₋₄₅ peptide before purification. Several peaks may represent the byproducts but the large peak is probably the target peptide. FIG. 12 shows a chromatogram of a prep HPLC run of HBD-3₃₆₋₄₅ peptide. The major peak likely contains the desired peptide. FIG. 13 shows a chromatogram of an analytical HPLC run of HBD-3₃₆₋₄₅ peptide after purification. The single peak indicates successful purification of the peptide.

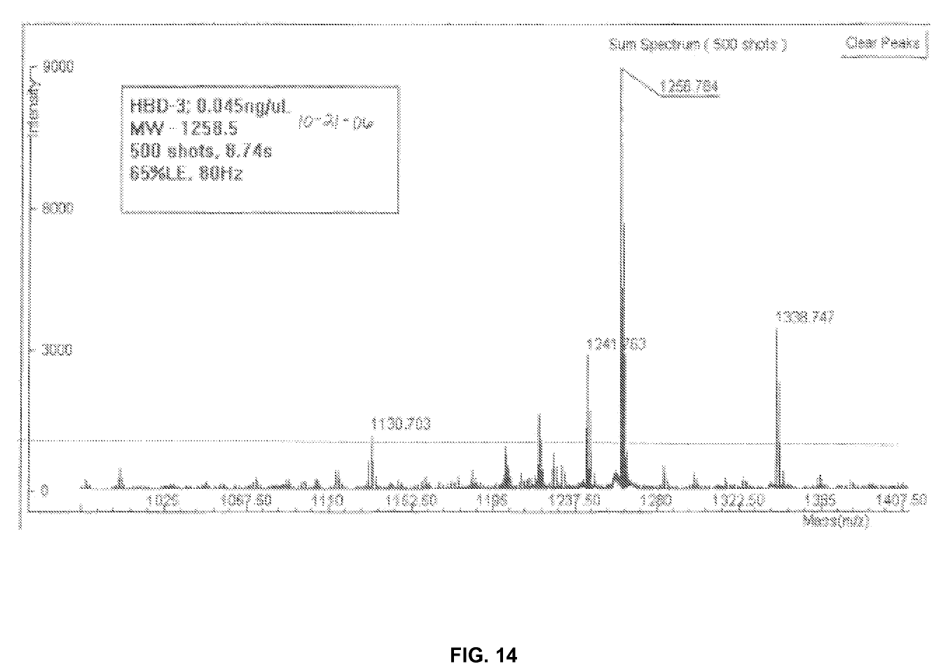

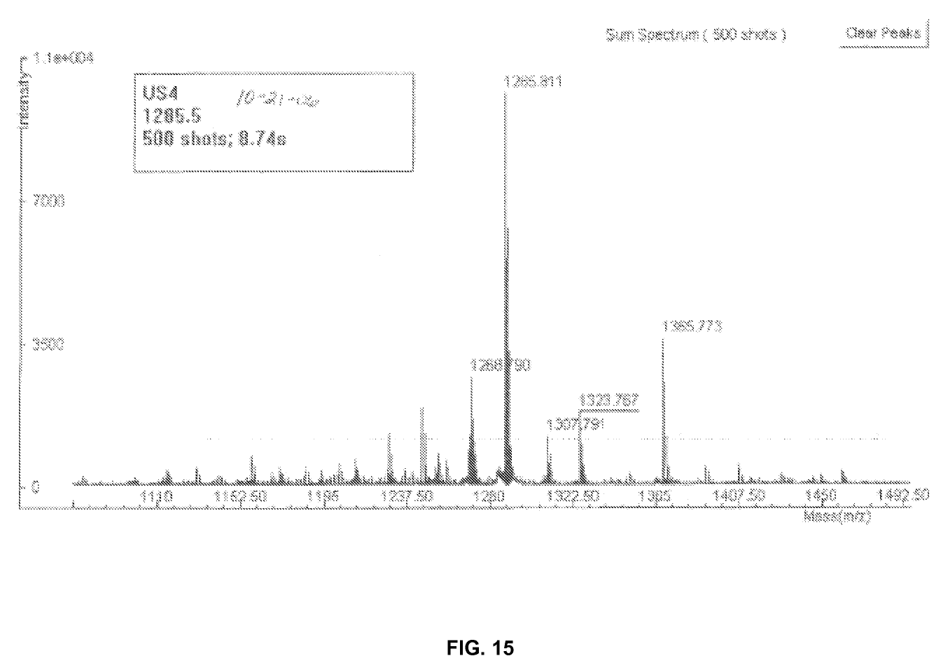

In identifying the peptides, matrix-assisted laser desorption/ionization—time of light (MALDI-TOF) mass spectrometry may be performed. The MALDI-TOF results, as shown in FIGS. 14 and 15, confirms the identification of HBD-3₃₆₋₄₅ and HBD-3₃₆₋₄₅(Rev2), respectively. The molecular weight of HBD-3₃₆₋₄₅ was determined to be about 1258.5 g/mol. The molecular weight of HBD-3₃₆₋₄₅(Rev 2) was determined to be about 1285.5 g/mol.

E. Conjugating Peptides to Cross-Linked Agarose

Peptides may be conjugated to agarose.

In one example, according to Pierce, derivatized cross-linked agarose (500 μl) may be washed several times with 100 mM phosphate buffer (pH ˜7.5). The target peptide (e.g., ˜2 mg) may then be dissolved in 600 μl of phosphate buffer. The agarose may be suspended in 500 μl of the peptide solution, and 5-7 μl of NaCNBH₃ may be added to the suspension. The reaction is allowed to rock gently over night at 4° C. The following morning, the reaction may be centrifuged in order to pellet the agarose, and the supernatant can be drawn off and set aside. The resin may be suspended in 500 μl of 1 M Tris buffer (pH ˜7.5), and 5-7 μl of NaCNBH₃ solution may be added to the suspension. This reaction is then allowed to rock gently at 4° C. for 1 hour. After this time, the Tris solution may be removed; the conjugated agarose may be washed repeatedly with water. The washed peptide-agarose conjugate may be stored as a 50% v/v in water at 4° C. The degree of loading of the agarose may be estimated by comparing the absorption at 220 nm of the initial peptide solution to that of the solution after conjugation. This protocol has been shown to consistently afford peptide-agarose conjugates with peptide loading of 3-4 mg of peptide/ml of agarose.

In another example, the peptide HBD-3₃₂₋₄₅ (approximately 2.5 mg) may be dissolved in 600 μl of phosphate buffer (˜100 mM Na₂HPO₄, titrated to pH ˜7.5). Agarose beads (˜1 ml of a 50% suspension in 20% ethanol) may be centrifuged for 2 min. at 3,300 rcf. After centrifugation, the supernatant may be removed. The resin may be resuspended in 1 ml of phosphate buffer. The agarose may be centrifuged again for 2 min. at 3,300 rcf, followed by removal of the supernatant. This process may be repeated (e.g., 4 more times). After completing multiple repetitions, the agarose may be suspended in 500 μl of the peptide solution. The remaining 100 μl of peptide solution can be set aside and stored at 4° C. The reducing agent NaCNBH₃ (10 μl of a 1 M solution) may be added to the suspended agarose, and the reaction may be incubated at 4° C. overnight with agitation. The reaction mixture may be centrifuged for 2 min at 3,300 rcf. To eliminate unreacted aldehyde groups present on the agarose, the resin may be suspended in 500 μl of 1 M Tris having a pH of about 7.5. About 10 μl of the NaCNBH₃ solution may be added to the suspension. The reaction may be allowed to continue for 30 minutes at room temperature and then centrifuged at 3,300 rcf for 2 min. Once the supernatant is removed, the peptide conjugate may be washed with 1 M NaCl (e.g., three times, 500 μl each) as described earlier in order to remove uncoupled peptides and other reagents. The resin may be washed with sterile water (e.g., three times, 500 μl each). Following the washes, the conjugated agarose may be suspended in 500 μl of sterile water and stored at 4° C.

In another example, phosphate buffer (50 ml at 0.1 M, pH ˜7) may be prepared and sterile filtered. Cross-linked agarose resin (1 ml of a 50% suspension) may be transferred to a 1.5 ml microcentrifuge tube. The suspended resin may be centrifuged for 2 minutes at 3,300 rcf. After centrifugation, the supernatant may be removed. The resin may be suspended in 0.9 ml of sterile phosphate buffer, and then centrifuged for 2 minutes at 3,300 rcf. The supernatant may be removed again, and the process may be repeated (e.g., 4 times). Purified HBD-3₃₆₋₄₅ (3.9 mg) may be dissolved in 1 ml of sterile phosphate buffer, and the prepared resin (0.433 ml) may be resuspended in 0.5 ml of the peptide solution. Aqueous NaCNBH₃ (5 μl of a 1 M solution) may be added to the mixture and the reaction was allowed to incubate at 4° C. with agitation. After incubation (e.g., overnight), the combination may be centrifuged for 2 minutes at 3,300 rcf, and the supernatant may be collected. The resin may be resuspended in 500 μl of Tris buffer (1 M, pH ˜7.5), and 5 μl of NaCNBH₃ solution may be added to the reaction (to block any remaining free aldehyde groups on the resin). The reaction can be incubated for 1 hr at room temperature with agitation. After the allotted time, the reaction may be centrifuged for 2 minutes at 3,300 rcf; the supernatant may be removed. The resin may be washed repetitively (e.g., 3 times) with 1 M NaCl, followed by three washes with sterile distilled deionized (ddi) water as described in the initial resin preparation. After the final wash, the pelleted resin may be suspended in 500 μl of ddi water and stored at 4° C. This procedure may provide a peptide-agarose conjugate with a loading of ˜3.0 mg/ml. Loading estimate can be based on comparison of the AU₂₂₀ of the peptide solution before and after conjugation.

In another example, cross-linked agarose has been chemically modified, and aldehyde groups have been incorporated into the polysaccharide. Using FIG. 4 for exemplified, illustrative purposes, aldehyde groups react with primary amines to form imines, which can be readily reduced to amines under mild conditions. Here, peptides or peptide mimetics may be combined with agarose in 100 mM sodium phosphate and 100 mM NaCl. The pH may be approximately 7.4. The reducing agent, NaCNBH₃, may then be added to the mixture. The reaction is then generally incubated at room temperature with agitation for ˜6 hours. After the desired time has elapsed, the solution may be separated from the agarose. The beads may then be treated with 1 M Tris, with a pH ˜7. Afterwards, NaCNBH₃ may once more be added. The reaction is allowed to continue for ˜1 hour, and the resin can then be washed with 1 M NaCl. Treating the resin with 1 M Tris may block aldehyde groups, which are not already occupied by peptides, to prevent possible side reactions.

Peptide loading can be estimated by comparing the UV absorption at, for example, 280 nm of the phosphate solution containing a known amount of peptide before adding resin to the combined phosphate-peptide solution after incubation with resin. The difference in readings can then be correlated to the amount of peptide loaded onto the resin. Some of the peptides may not have adequate absorption at 280 nm for this approach. Therefore, alternative wavelengths (220 or 230 nm) and HPLC analysis may need to be conducted.

At 220 nm, peptide loading can be estimated by first preparing a solution of 2.5 mg of peptide in 600 μl of phosphate buffer (100 mM, pH ˜7.5). About 100 μl of the solution may be set aside for UV analysis (before incubation); 500 μl of it may be conjugated with agarose. The AU₂₂₀ of the peptide solution before reaction with agarose may be 0.265. The AU₂₂₀ of the peptide solution after incubation may be 0.022, which corresponds to a signal loss of 91.7%. Before conjugation there may be approximately 2.083 mg of peptide in 500 μl of buffer (2.5 mg of peptide/600 μl of phosphate=0.00417 mg peptide in 1 μl of solution). The loading of HBD-3₃₂₋₄₅ peptide can be estimated to be about 3.82 mg of peptide per ml of resin. The peptide loading of HBD-3₃₆₋₄₅ can be estimated to be about 3.0 mg/ml using this approach. The corresponding peptide conjugates may be assayed using E. coli (BL21 (DE3)).

Peptide concentration, pH, reaction times, and temperatures can be adjusted to maximize reaction efficiency and loading of peptide. Alternative blocking strategies can be used where Tris is replaced by another small molecule with an amino group, such as glycine, phenylalanine, serine, lysine, aspartate, etc.

Peptides may be bound to the polysaccharide in multiple orientations because this chemistry does not readily differentiate between N-terminal amino groups and those on the side-chains of lysine residues. It is assumed that the antimicrobial activity of a peptide-polysaccharide/mimetic conjugate is likely to be the result of a bulk property, and that a sufficient amount of bound peptides/mimetics will likely be in an orientation appropriate for the conjugate to kill target microbes.

F. Oxidation of Dextran

In addition to agarose, other polysaccharides may be used to create peptide-polysaccharide conjugates. In one embodiment, dextran may be used. However, it may be necessary to present aldehyde groups onto dextran to allow for Schiff base formation.

These functional groups may be introduced to dextran by, for instance, dissolving dextran (approximately 0.5 g, 100 to 200 kDa) in 7 ml of water while being cooled in an ice bath. Sodium periodate (˜1.17 g) may be added into the solution; the reaction can be allowed to stir in an ice bath for about 90 min. The tube containing the reaction mixture may be covered with aluminum foil during the course of the reaction because sodium periodate tends to be light sensitive. After stirring, the solution may desalt (to remove sodium periodate) with PD-10 Desalting column. The first flow thru may be collected, and then the column may be washed three times with 2 ml of water. The flow thru from every wash may be collected. It is likely that crystals are observable in tubes containing the first flow thru and the second flow thru. About 100 μl of oxidized dextran (from each tube containing crystals) can be concentrated for further analysis.

G. Functionalizing and/or Conjugating Peptides to Oxidized Cellulose Peptides may also be functionalized and/or conjugated to cellulose. Similar to dextran, functional groups (i.e., aldehyde groups) may need to be introduced to cellulose to allow for Schiff base formation.

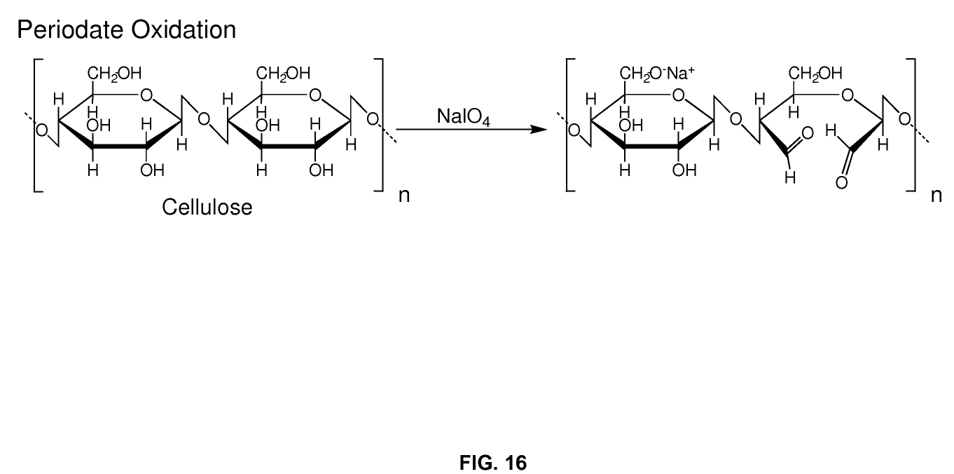

In one embodiment, periodate oxidation can be used to introduce aldehyde groups into cellulose via oxidative cleavage of the bond between carbons 2 and 3 of glucose residues in the polysaccharide. As illustrated in FIG. 16, this introduction may result in the formation of two aldehyde groups, namely one on the 2 carbon and the other on the 3 carbon. Periodate oxidation is typically relatively mild and is a powerful tool for functionalizing both soluble and insoluble polysaccharides.

While periodate oxidation may not directly affect the glycosidic bonds of cellulose, it tends to result in the cleavage of a carbon-carbon bond (e.g., within some glucopyranose residues) and the formation of two aldehyde groups. This breaking may compromise some of the stability that is normally associated with cellulose.

One way of conducting periodic oxidation is by suspending porous cellulose beads in aqueous NaIO₄. The suspension may be allowed to mix gently at room temperature in the dark. After a proscribed reaction time, ethylene glycol may be added to the mixture to terminate the oxidation of cellulose. The oxidized cellulose may then be washed repeatedly with water. Depending on stoichiometry, approximately 10-40% of glucose residues may be oxidized.

The degree of oxidation can be evaluated by treating a small portion of the cellulose with a large excess of lysine to form imine. Imine can then be reduced by adding NaCNBH₃. The primary amine content can be determined by using quantitative Kaiser assays. There should be two amino groups present for every glucose residue that has been oxidized. Thus. it is possible to determine the aldehyde content, mmol/g of cellulose. The degree of oxidation can be controlled by varying the molar equivalents of NaIO₄ used per gram of cellulose. Reaction times, pH and temperatures can also be altered in order to tailor the degree of functionalization.

One alternative to modifying cellulose deals with the likelihood of selectively oxidizing the 6-hydroxyl group on cellulose glucose residues under mild conditions using TEMPO and NaOCl. This selective oxidation may provide a similar aldehyde. However, it may de difficult to prevent over-oxidation to the carboxylic acid.

A second alternative is introducing a maleimide containing linker by forming an ester linkage between the carboxyl group of the linker hydroxyl groups of cellulose. Maleimides are reactive towards thiol groups, such as that of cysteine residues. A common strategy for conjugating or functionalizing proteins, this introduction is likely to produce a thioether.

As another embodiment, cellulose may be oxidized to obtain 10% oxidation using the exemplified protocol. Approximately 500 mg of cellulose (cellulose, powder, ˜20 micron) may be placed inside a 50 ml round bottom flask. Once inside the flask, ˜15 ml of distilled water may be added to suspend the cellulose. The mixture may then be stirred using, for example, a magnetic stirrer and stirring plate. Because the reaction tends to be light sensitive, the flask is loosely stoppered and wrapped in aluminum foil. Approximately 351 mg of sodium (meta) periodate (˜1.64 mmols) may be added and the reaction is allowed to stir at room temperature for about 90 mins. After the reaction takes place, the oxidized polysaccharide may be pelleted by centrifuging at 3000 rpm, the supernatant may be removed, and the cellulose may be washed with distilled water and again centrifuged. This washing should be repeated numerous times (e.g., 3 more times) to remove excess sodium (meta) periodate. After repeated washes, the polysaccharide may be suspended in 5 ml of distilled water. This polysaccharide stock solution may be stored, kept refrigerated (e.g., at 3.4° C.), and later implemented in an oximation reaction with hydroxylamine.

Determining the degree of oxidation may based on the exemplified following. The oximation reaction of oxidized cellulose and hydroxylamine may be accomplished with 3% polysaccharide stock suspension and 0.4 M hydroxylamine (NH₂OH.HCl) stock solution may be used. About 100 μl of 3% polysaccharide solution, about 300 μl of 0.4 M hydroxylamine solution, and about 2.6 ml of distilled water may be placed into a 15 ml falcon tube. The reaction mixture may then be placed in a 42° C. water bath and allowed to react for about 2 hrs. After the reaction takes place, the mixture may then be titrated using the following steps.

The initial pH of the NH₂OH.HCl solution may be measured using a pH meter. A control comprising a hydroxylamine solution and ˜100 μl of water may be created, and the pH of the control solution may also be measured (pH ˜3.74). The control pH may serve as a reference point for when the base is consumed by the HCl liberated with oxime formation.

Approximately 0.1 N NaOH stock solution may be created as a base for titration. The NaOH solution may then be added in 2 μl aliquots to the NH₂OH.HCl solution, and the pH may be measured. This process may be repeated until a neutral pH of ˜7.4 is reached.

Data obtained from the titration may be plotted as pH v. Volume of base added. The resulting graph may be used to determine the volume required to reach a pH of ˜3.74. since the amount of base added is typically proportional to the amount of aldehyde groups present, the percent oxidation can be determined, in which for this experiment is around 10%.

Peptides may be conjugated to cellulose using the same general method as described for agarose conjugation. Since the cellulose is a finer particle size than is the agarose originally used, oxidized cellulose does not lend itself to measurement by volume. Instead, peptide loading is given in terms of mg peptide/mg of cellulose. In a preliminary conjugation reaction using Rev2, a loading of 3.2 milligrams of peptide per gram of cellulose was achieved.

The process for coupling peptides and β-peptides resembles that used with agarose. Peptides and mimetics can be incubated in phosphate buffer with functionalized cellulose and NaCNBH₃. The unreacted aldehyde groups may then be capped by incubating with 1 M Tris or glycine. As with the agarose conjugations, reaction conditions (such as peptide/mimetic concentration, reaction times, temperatures, etc.) can be adjusted to alter the loading of the cellulose.

The solubility properties of peptide conjugates may be different from that of their agarose counterparts, which are internally cross-linked and are insoluble in aqueous buffer. Should the cellulose derivatives and peptide/mimetic conjugates be soluble in aqueous buffer, Tris or glycine can be added directly to the peptide coupling reaction after it has been allowed to go to completion. The resulting conjugate can then be separated from the unbound peptide by FPLC and gel filtration.

H. Antimicrobial Activity

Overall, antimicrobial activity can be evaluated by incubating small cultures of E. coli (BL21) at 37° C. with agitation (e.g., ˜3 to ˜5 hrs) in minimal media in the presence/absence of varied amounts of peptide conjugate or free peptide. Aliquots can be collected at predetermined time points to determine the cell density. One OD₆₀₀ of the solution=2×10⁹ CFU/ml. Correlating changes in cell density between cultures incubated with varied amounts of conjugate and cultures grown in the absence of conjugates can provide a means of evaluating the antimicrobial potency of the varied constructs. Assays such as this one are known to be used in evaluating the potency of antimicrobial peptidomimetics and biomaterials.

In one embodiment, the antimicrobial activity of the free peptides and conjugates have been assayed against E. coli BL21(DE3) cultures. In this assay, the bacteria (at ˜1×10⁶ CFU/ml) are incubated for 2 hours at 37° C. in 10 mM phosphate (e.g., Na₂HPO₄) buffer (pH ˜7.5) with varied concentrations of peptide or peptide-conjugate. After this time, 20 μl aliquots of each culture and serial dilutions of the culture are plated onto LB agar plates and incubated over night at 37° C. The number of colonies on these plates is then counted and the survival percentage is determined relative to a control culture. The example contained no peptide.

In another embodiment, a stock solution containing 1 mg of peptide per ml solution may be prepared. Using the stock peptide (1 mg/ml) solution, assays may be prepared (volume of ˜100 μl) at 1 μg/ml, 10 μg/ml, and 100 μg/ml of free peptide (e.g., HBD-3₃₂₋₄₅). In the control sample, 10 μl of 100 mM phosphate buffer, 80 μl of sterile water and 10 μl of cell cultures may be combined and incubated without peptide for two hours at 37° C. After two hours incubation, 20 μl aliquots of the above solutions, and 1:10, and 1:100 dilutions of each may be plated in LB-agar plates and incubated over night at 37° C.

In another embodiment, peptide conjugate (e.g., HBD-3₃₂₋₄₅-agarose) with a loading of 3.82 μg of peptide/1 μl of resin may also be assayed at effective peptide concentrations of 1 μg/ml, 10 μg/ml, 25 μg/ml, 50 μg/ml, 75 μg/ml and 100 μg/ml. For example, an effective peptide concentration of 100 μg/ml of suspension may be prepared by using 52.2 μl of a 50% suspension of 3.82 μg/μl of peptide suspension (100 μg×[1 μl/3.82 μg]=26.1 μl). Samples containing varying concentrations of peptide conjugate may be combined with 10 μl of 100 mM phosphate buffer having a pH of about 7.5, 10 μl of cell cultures (˜1×10⁷ CFU/ml), and the required volume of sterile water to bring a total volume of 100 μl. This combination may be incubated at 37° C. with agitation for two hours. After incubation, 20 μl of each cell culture and 1:10 and 1:100 dilutions of each cell culture may be plated on LB-agar plates and incubated at 37° C. overnight. Similar protocols may be used to evaluate the antimicrobial activity of the HBD-3₃₆₋₄₅ peptide and corresponding conjugate.

In another embodiment, antimicrobial assays may be carried out using dissolved free HBD-3₃₆₋₄₅ and suspended conjugate. LB (50 ml) may be inoculated with 1 ml of a BL21 starter culture. The new culture may be allowed to grow at 37° C. until reaching an OD₆₀₀ between 1 and 2. This stock may then be diluted with sterile water to provide a culture of 1×10⁷ cfu/ml. About 100 μl cultures of E. coli BL21 (˜1×10⁶ cfu/ml) may be incubated for two hours at 37° C. in 100 mM phosphate, having a pH ˜7.8, in the presence of 1 μg/ml of free peptide, 10 μg/ml of peptide, the equivalent of 10 μg/ml of peptide-conjugate, 100 μg/ml conjugate, 1 mg/ml conjugate and with no peptide or conjugate present. Serial dilutions of these cultures may then be plated on LB-agar plates and incubated overnight at 37° C.

It should be noted that assays for some of the free peptides (Rev2 and HBD-3₃₆₋₄₅) may be done in triplicates.

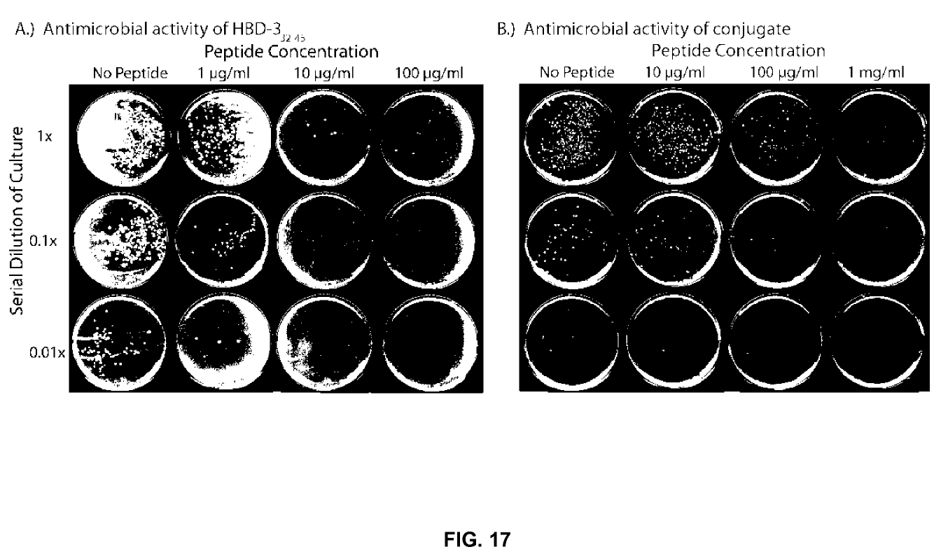

The results of the antimicrobial assay using free HBD-3₃₂₋₄₅ and agarose conjugate can be seen in FIG. 17. Antimicrobial activity of free HBD-3₃₂₋₄₅ is given on the left and that of the HBD-3₃₂₋₄₅ peptide-conjugate is shown on the right. E. coli at 1×10⁶ CFU/ml were incubated with varied peptide concentrations (free peptide and peptide conjugate). Serial dilutions (0.1× and 0.01×) of cell cultures in all plates are shown across the figure.

The free peptide killed ˜93% of bacteria at a peptide concentration of 1 μg/ml. At concentrations of 10 μg/m and 100 μg/ml, microbicidal efficiency was >99%. At an effective peptide concentration of 10 μg/ml, the peptide-agarose conjugate appeared to kill ˜50% of the bacteria present in the culture. A significantly greater proportion of cells appear to be killed at higher effective peptide concentrations (e.g., 100 μg/ml and 1 mg/ml).

HBD-3₃₂₋₄₅ peptide-agarose conjugate may also be assayed at effective peptide concentrations of 25 μg/ml, 50 μg/ml, and 75 μg/ml, to more accurately estimate the 90% lethal concentration (“LC₉₀”). Based on the results of these additional assays, it is possible to estimate the LC₉₀ of the peptide-agarose conjugate (˜75 μg/ml). TABLE 3 reflects results of the antimicrobial assays.

With respect to HBD-3₃₆₋₄₅, the free peptide of HBD-3₃₆₋₄₅ in the antimicrobial assay caused ˜67% reduction in the number of colonies at a peptide concentration of 1 μg/ml and ˜97% reduction at the concentration of 10 μg/ml. The peptide-agarose conjugate also produced a reduction in the number of colonies, but it appears to require a higher concentration to be effective. At an effective peptide concentration of 1 mg/ml, the conjugate brought about ˜66% reduction in the number of colonies. The unmodified agarose did not appear to demonstrate any antimicrobial ability in this assay.

TABLE 3
Antimicrobial Activity Summary

| | | Reported Potency: | Measured Potency: | Measured Potency: |
|---|---|---|---|---|
| | | Free Peptide^{‡} | Free Peptide^{‡} | Peptide/Agarose |
| Peptide | Amino Acid Sequence | (LC₉₀) | (LC₉₀) | Conjugate^{‡} (LC₉₀) |
| HBD-3₃₂₋₄₅ (14-mer) | KSSTRGRKSSRRKK | ~1 μg/ml | 0.20 μg/ml | ~75 μg/ml |
| | | | | ~10 μg/ml (LC₅₀) |
| HBD-3₃₆₋₄₅ (10-mer) | RGRKSSRRKK | ~4 μg/ml | 2.23 μg/ml | >100 μg/m |
| HBD-3₃₆₋₄₅ (Rev 2) | RGRRSSRRKK-NH₂ | N/A | 0.15 μg/ml | ~26 μg/ml |

^{‡}Antimicrobial potency test against E. coli (BL21 (DE3).

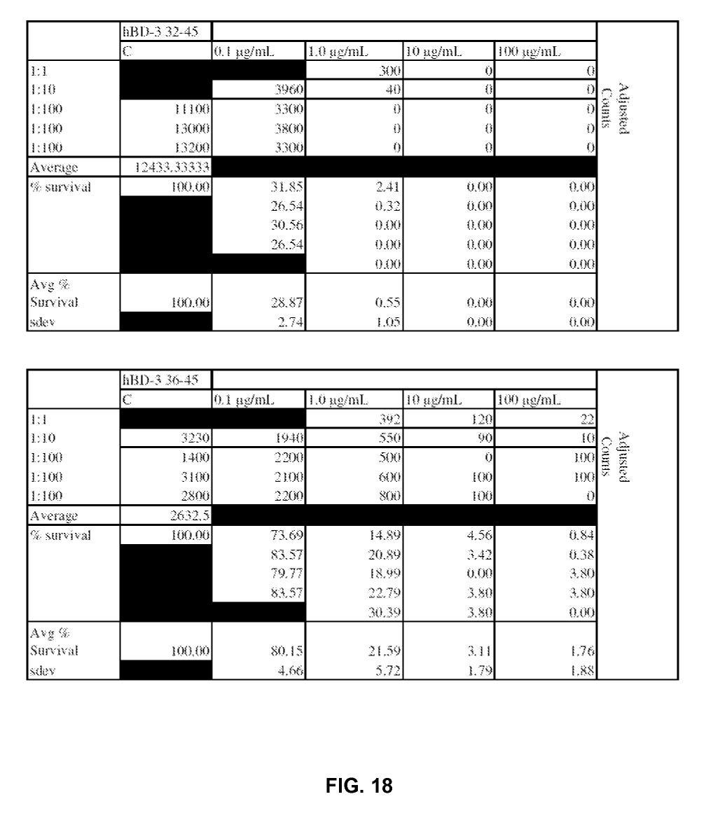

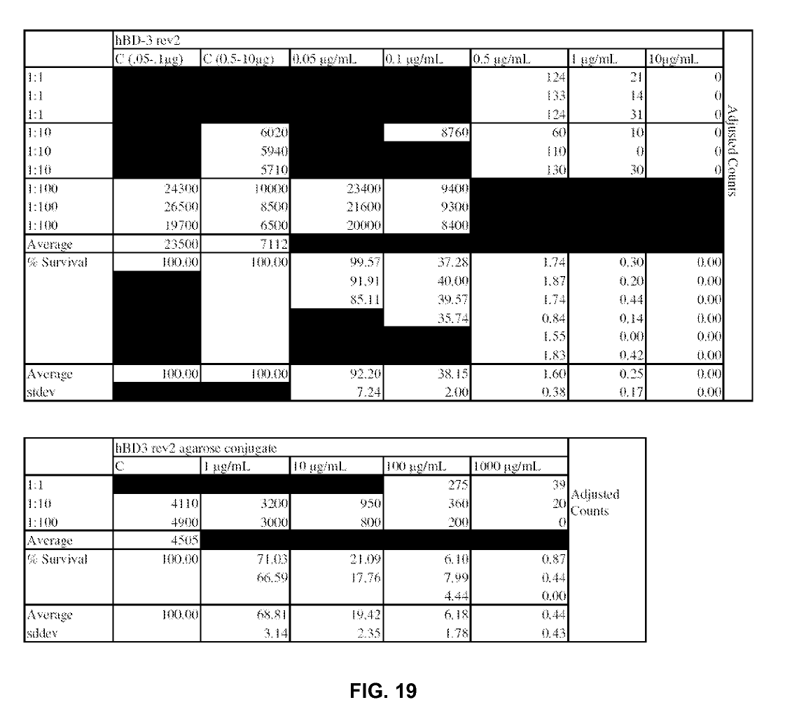

Results of E. coli colony counts may be seen in FIGS. 18 and 19. In particular, the top table in FIG. 18 uses peptide HBD-3₃₂₋₄₅. The bottom table in FIG. 18 uses peptide HBD-3₃₆₋₄₅. The top table in FIG. 19 uses peptide HBD-3₃₆₋₄₅(Rev2). The bottom table in FIG. 19 uses peptide-polysaccharide conjugate HBD-3₃₆₋₄₅(Rev2)—agarose conjugate. Each of these four tables shows E. coli colony count results for various dilution ratios (i.e., 1:1, 1:10 and 1:100), concentration of either peptide or peptide-polysaccharide conjugate, and survival percentages, where E. coli was tested against a particular peptide and/or peptide-polysaccharide conjugate.

When the 10-residue peptide HBD-3₃₆₋₄₅ is compared with the 14-residue peptide HBD-3₃₂₋₄₅, it is likely to demonstrate lower antimicrobial activity. One possible reason for the lower activity may be the fact that HBD-3₃₆₋₄₅ has 4 less amino acids than HBD-3₃₂₋₄₅, and that these 4 residues may contribute in some way to the microbicidal mechanism. It may also provide a spacer separating the polysaccharide from the peptides conjugated through their N-terminus. The details of the antimicrobial mechanism used by HBD-3₃₂₋₄₅ and HBD-3₃₆₋₄₅ to attack bacteria is not apparently known and is likely different from that of the parent peptide. There is also the possibility that the lack of the 4 amino acids in HBD-3₃₆₋₄₅ may cause an important structure change and cause HBD-3₃₆₋₄₅ to be less potent than HBD-3₃₂₋₄₅.

The results of these studies suggest that both the free peptide and the peptide-agarose conjugate show good antimicrobial activity, with the conjugate being less potent than the free peptide. There are several possible explanations for the peptide-conjugate showing less potency than the free peptide. The peptides may be bound to the resin in an unproductive orientation. Peptides may also be attached at sites in the interior space of the agarose, which may result in peptides not being exposed to bacteria. Since agarose beads tend to be insoluble, it may have resulted in cells not being adequately mixed or exposed to peptides. Additionally, there may be multiple points of attachment of peptide to resin because the resin tends to link to the peptide via the primary amine; there are four lysines that have a primary amine in the sequence. Furthermore, the same peptide may be linking several times to the agarose, thus affecting its antimicrobial ability. While the conjugate shows lower potency than the free peptide, these results are still encouraging, because attachment of the peptide to the polysaccharide does not necessarily eliminate or drastically decrease the potency of the bound peptide.

In another embodiment, antimicrobial assays such as these may be repeated using other microbes. An example of such microbe is the K12 strain of E. coli. This E. coli strain may provide a better target for evaluating antimicrobial activity. These assays can be carried out using conditions based on those described above. Furthermore, these assays may be carried out in triplicate in order to get more precise values for the antimicrobial activities of the conjugates. Moreover, these studies may be extended to include other attached polysaccharides and their derivatives.

In another embodiment, antimicrobial activity may be evaluated by incubating E. coli (BL21) at 37° C. in the presence/absence of suspended peptide conjugate. In these assays, varied amounts of conjugate may be suspended in sterile phosphate (pH ˜7), forming a thick suspension or slurry. After approximately two hours, the reaction can be diluted with 100 μl of sterile water. Aliquots of the prepared sample may be serially diluted and plated onto LB-agar plates. The plates may then be incubated overnight at 37° C. After this incubation, the colonies on each of the plates can be counted. Similar protocols are also used to determine the antimicrobial activity of the free peptides. By comparing the performance of conjugates, free peptides, and control samples containing no antimicrobial agents, one may be able to evaluate the relative antimicrobial potencies of engineered constructs.

I. Hemolytic Activity

Hemolytic activity may be evaluated using, for example, murine erythrocytes. Varied amounts of conjugate may be added to the cells suspended in 10 mM Tris and 150 mM NaCl. The pH may be about 7.0. The mixture can then be incubated at 37° C. for 1 hour. The culture can be centrifuged to pellet the suspended cells. The supernatant may be collected to determine the OD₄₁₄. Absorption at 414 nm is likely due to released hemoglobin. The degree of hemolysis can be determined by comparing the results for each sample to the OD₄₁₄ of cells incubated with melitin (e.g., about 50 μM), which may provide a reference point for complete hemolysis. Plotting the percent hemolysis for each construct as a function of the amount of conjugate may provide a measure of hemolytic activity. This protocol may be used to evaluate the hemolytic activity of antimicrobial β-peptides.

The foregoing descriptions of the embodiments of the claimed invention have been presented for purposes of illustration and description. They are not intended to be exhaustive or be limiting to the precise forms disclosed, and obviously many modifications and variations are possible in light of the above teaching. The illustrated embodiments were chosen and described in order to best explain the principles of the claimed invention and its practical application to thereby enable others skilled in the art to best utilize it in various embodiments and with various modifications as are suited to the particular use contemplated without departing from the spirit and scope of the claimed invention. In fact, after reading the above description, it will be apparent to one skilled in the relevant art(s) how to implement the claimed invention in alternative embodiments. Thus, the claimed invention should not be limited by any of the above described example embodiments. For example, the claimed invention may be practiced over other animals (such as treatment of domestic animals in veterinary clinics).

In addition, it should be understood that any figures, graphs, tables, examples, etc., which highlight the functionality and advantages of the claimed invention, are presented for example purposes only. The architecture of the disclosed is sufficiently flexible and configurable, such that it may be utilized in ways other than that shown. For example, the steps listed in any flowchart may be reordered or only optionally used in some embodiments.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the claimed invention of the application. The Abstract is not intended to be limiting as to the scope of the claimed invention in any way.

Furthermore, it is the applicants' intent that only claims that include the express language “means for” or “step for” be interpreted under 35 U.S.C. §112, paragraph 6. Claims that do not expressly include the phrase “means for” or “step for” are not to be interpreted under 35 U.S.C. §112, paragraph 6.

A portion of the claimed invention of this patent document contains material which is subject to copyright protection. The copyright owner has no objection to the facsimile reproduction by anyone of the patent document or the patent invention, as it appears in the Patent and Trademark Office patent file or records, but otherwise reserves all copyright rights whatsoever.

What is claimed is:

1. A peptide-polysaccharide conjugate comprising at least one peptide linked with a polysaccharide, peptide being a defensin and said peptide being capable of exerting antimicrobial activity.

2. A method according to claim 1, wherein said defensin comprises at least one peptide based on at least a portion of the amino acid sequence of human β-defensin-3, said portion being the last 10-14 residues of said amino acid sequence.

3. A peptide-polysaccharide conjugate according to claim 2, wherein said peptide has a partial amino acid sequence comprising KSSTRGRKSSRRKK (SEQ ID NO. 2).

4. A peptide-polysaccharide conjugate according to claim 2, wherein said peptide has a partial amino acid sequence comprising RGRKSSRRKK (SEQ ID NO. 3).

5. A peptide-polysaccharide conjugate according to claim 2, wherein said peptide has a partial amino acid sequence comprising RGRRSSRRKK (SEQ ID NO. 4) and an amide group located on the C-terminus.

6. A peptide-polysaccharide conjugate according to claim 2, wherein the site of attachment of said peptide for said polysaccharide comprises at least one of the following:
a. the ε-amino group of lysine of said peptide; and
b. the α-amino group of the N-terminus of said peptide.

7. A peptide-polysaccharide conjugate according to claim 1, wherein said polysaccharide is cellulose.

8. A peptide-polysaccharide conjugate according to claim 1, wherein said polysaccharide is agarose.

9. A peptide-polysaccharide conjugate according to claim 1, wherein said polysaccharide is functionalized via at least one R group prior to linking with said peptide, said at least one R group including:
a. aldehyde;
b. amine;
c. carboxylic acid;
d. hydroxyl;
e. ester;
f. thiol;
g. halide;
h. halide-equivalent; and
i. epoxide.

10. A peptide-polysaccharide conjugate according to claim 9, wherein a reducing agent is used to stabilize the linkage formed between said peptide and said polysaccharide, said polysaccharide being functionalized with said at least one R group being said aldehyde.

11. A method of producing a peptide-polysaccharide conjugate comprising linking a peptide with a polysaccharide to generate said peptide-polysaccharide conjugate, said peptide being a defensin and said peptide being capable of exerting antimicrobial activity.

12. A method according to claim 11, wherein said defensin comprises at least one peptide based on at least a portion of the amino acid sequence of human β-defensin-3, said portion being the last 10-14 residues of said amino acid sequence.

13. A method according to claim 12, wherein said peptide has a partial amino acid sequence comprising KSSTRGRKSSRRKK (SEQ ID NO. 2).

14. A method according to claim 12, wherein said peptide has a partial amino acid sequence comprising RGRKSSRRKK (SEQ ID NO. 3).

15. A method according to claim 12, wherein said peptide has a partial amino acid sequence comprising RGRRSSRRKK (SEQ ID NO. 4) and an amide group located on the C-terminus.

16. A method according to claim 12, wherein the site of attachment of said peptide for said polysaccharide comprises at least one of the following:
a. the ε-amino group of lysine of said peptide; and
b. the α-amino group of the N-terminus of said peptide.

17. A method according to claim 11, wherein said polysaccharide is cellulose.

18. A method according to claim 11, wherein said polysaccharide is agarose.

19. A method according to claim 11, further including functionalizing said polysaccharide via at least one R group prior to linking with said peptide, said at least one R group including:
i. aldehyde;
ii. amine;
iii. carboxylic acid;
iv. hydroxyl;
v. ester;
vi. thiol;
vii. halide;
viii. halide-equivalent; and
ix. epoxide.

20. A method according to claim 19, further including adding a reducing agent to said peptide-polysaccharide conjugate for stabilizing the linkage formed between said peptide and said polysaccharide, said polysaccharide being functionalized with said at least one R group being said aldehyde.


SEQUENCE LISTING (4 sequences)

SEQ ID NO: 1 - protein, 45 aa, Homo sapiens
  PEPTIDE (32)..(45): Human beta defensin 3 (hereinafter referred to as HBD-3) X = C or S
GIINTLQKYYCRVRGGRCAVLSCLPKEEQIGKXSTRGRKXXRRKK

SEQ ID NO: 2 - protein, 14 aa, Homo sapiens
  PEPTIDE (1)..(14): Fragment of HBD-3, where state number 1 corresponds to state number 32 of Sequence ID number 1 X = C or S
KXSTRGRKXXRRKK

SEQ ID NO: 3 - protein, 10 aa, Homo sapiens
  PEPTIDE (1)..(10): Fragment of HBD-3, where state number 1 corresponds to state number 36 of Sequence ID number 1 X = C or S
RGRKXXRRKK

SEQ ID NO: 4 - protein, 10 aa, Homo sapiens
  PEPTIDE (1)..(10): Fragment of HBD-3, where state number 1 corresponds to state number 36 of Sequence ID number 1 K of state number 39 of Sequence ID number 1 is replaced with R X = C or S
RGRRXXRRKK